(19) United States
(12) Patent Application Publication
(10) Pub. No.: US 2022/0145335 A1
(43) Pub. Date: May 12, 2022

(54) Bio-catalyzed Synthesis of Potent Anti-inflammatory Agents from Medroxyprogesterone Acetate

(71) Applicants: Muhammad Iqbal Choudhary, Karachi (PK); Saira Bano, Karachi (PK); Atia-tul Wahab, Karachi (PK); Zaheer Ul-Haq, Karachi (PK); Sammer Yousuf, Karachi (PK); Almas Jabeen, Karachi (PK); Atta-ur Rahman, Karachi (PK)

(72) Inventors: Muhammad Iqbal Choudhary, Karachi (PK); Saira Bano, Karachi (PK); Atia-tul Wahab, Karachi (PK); Zaheer Ul-Haq, Karachi (PK); Sammer Yousuf, Karachi (PK); Almas Jabeen, Karachi (PK); Atta-ur Rahman, Karachi (PK)

(21) Appl. No.: 17/585,620

(22) Filed: Jan. 27, 2022

(51) Int. Cl. C12P 7/26; C12N 1/14; A61P 35/00

(52) U.S. Cl. CPC C12P 7/26; C12R 2001/785; A61P 35/00; C12N 1/14

(57) ABSTRACT

Biotransformation of medroxyprogesterone acetate (MPA) (1) with Cunninghamella blakesleeana (ATCC 8688) yielded five new analogues, i.e. 17α-acetoxy-6α-methylpregn-4-ene-3,11,20-trione (2), 17α-acetoxy-15β-hydroxy-6α-methylpregn-4-ene-3,11,20-trione (3), 17α-acetoxy-6β-hydroxy-6α-methylpregn-4-ene-3,11,20-trione (4), 17α-acetoxy-11β,15β-dihydroxy-6α-methylpregn-4-ene-3,20-dione (5), and 17α-acetoxy-6β,11β-dihydroxy-6α-methylpregn-4-ene-3,20-dione (6). In T-cell proliferation assay, metabolites 2, and 5 were found to be potent inhibitors with IC₅₀<0.5 μM, metabolite 6 showed a significant activity with IC₅₀=8.64±0.02 μM, while metabolites 3 and 4 were found to be moderately active with IC₅₀=41.59±8.14, and 40.14±0.12 μM, as compared to substrate 1 (IC₅₀=6.48±5.18 μM) and standard prednisolone (IC₅₀=9.75±0.03 μM) in in vitro assay. To establish the binding mode of medroxyprogesterone acetate (MPA) and the bio-transformed derivatives, molecular docking simulations were carried out using Vina.

### BACKGROUND OF THE INVENTION

Medroxyprogesterone acetate (MPA) (1) is a synthetic progesterone that is commonly used as a contraceptive drug in human. MPA (1) is also widely used at higher doses in hormone replacement therapy (HRT) by women worldwide. It is commonly used in endocrine therapy for advanced or recurrent breast and endometrial cancers. MPA (1) is also reported for its anti-inflammatory effects [Young et al., J. Med. Primatol. 2021, 50, 51; Ugrasa et al., Mol. Cell. Endocrinol. 2021, 525, 111180; Lambrinoudaki, Case Rep. Womens Health. 2021, 29, e00270, Elovitz et al., Am. J. Obstet. Gynecol. 2004, 190, 693].

MPA (1) undergoes extensive and rapid metabolism in humans, and in experimental animals. The drug is extensively metabolized in the intestinal mucosa and in the liver. Cytochrome P450s (CYPs), involved in the metabolism of MPA, were identified by using human liver microsomes and recombinant human CYPs. Three major metabolites 6β-, 2β-, and 1β-hydroxy MPA have been reported by cytochrome P450s (CYPs) [Mimuraa et al., Life Sci. 2003, 73, 3201; Chen et al., Chem. Pharm. Bull. 2009, 57, 835].

Microorganisms are well known for their ability to catalyze whole range of organic compounds. As a result, microorganisms and their enzymes are widely employed in the synthesis of organic compounds, and modification of their structures. Structural transformation of steroidal compounds through microorganisms has emerged as an important approach in steroidal drug industry [Zappaterra et al., Molecules 2021, 26, 1352; Aziz et al., Steroids, 2020, 154, 108467; Siddiqui et al., Phytochem. Lett. 2021, 44, 137; Choudhary et al., Front. Pharmacol. 2017, 8, 900; Smith et al., Steroids. 2015, 102, 39].

Only the 11α-hydroxylation of medroxyprogesterone acetate (MPA) (1) by Absidia griseolla var. iguchii and Acremonium chrysogenum have been reported previously. Therefore, it is necessary to identify more microorganism for the production of polar derivatives of MPA, which have pharmaceutical applications. During this study, fermentation of MPA (1) was with Cunninghamella blakesleeana led to the formation of various oxidative metabolites which were found to possess anti-inflammatory activity in vitro [Ghasemi et al., Steroids 2019, 49, 108427].

### BRIEF SUMMARY OF THE INVENTION

In continuation of our research on microbial transformations, MPA (1) was incubated with Cunninghamella blakesleeana at ambient reaction conditions [Siddiqui et al., Phytochem. Lett. 2021, 44, 147; Chegaing et al., Steroids, 2020, 162, 108679; Siddiqui et al., J. Adv. Rev. 2020, 24, 69; Ibrahim et al., Steroids, 2020, 162, 108694; Aziz et al., Steroids, 2020, 154, 108467; Hussain et al., RSC Adv. 2020, 10, 451; Farooq et al., RSC Adv. 2018, 8, 21985; Atia-tul-Wahab et al., Bioorg. Chem. 2018, 77, 152; Choudhary et al., Front. Pharmacol. 2017, 8, 900; Siddiqui et al., PloS One, 2017, e0171476; Bano et al., PloS One. 2016, 11, e0153951]. This yielded five new metabolites. These metabolites were purified by high performance liquid chromatography (HPLC), and characterized as 17α-acetoxy-6α-methylpregn-4-ene-3,11,20-trione (2), 17α-acetoxy-15β-hydroxy-6α-methylpregn-4-ene-3,11,20-trione (3), 17α-acetoxy-6β-hydroxy-6α-methylpregn-4-ene-3,11,20-trione (4), 17α-acetoxy-11β,15β-dihydroxy-6α-methylpregn-4-ene-3,20-dione (5), and 17α-acetoxy-6β,11ββ-dihydroxy-6α-methylpregn-4-ene-3,20-dione (6) by using modern spectroscopic techniques.

In T-cell proliferation assay, compounds 2 and 5 were found to be potent inhibitors with IC₅₀<0.5 μM. Compound 6 showed a strong activity with IC₅₀=8.64±0.02 μM, while compounds 3 (IC₅₀=41.59±8.14 μM), and 4 (IC₅₀=40.14±0.12 μM) were found to be moderately active as compared to substrate (1) (IC₅₀=6.48±5.18 μM) and standard prednisolone (IC₅₀=9.75±0.03 μM) in vitro.

### BRIEF DESCRIPTION OF THE DRAWINGS

FIG. 1 depicts the structures of medroxyprogesterone acetate (MPA) (1), and its new metabolites 2-6 via Cunninghamella blakesleeana-mediated transformation of drug 1, along with their anti-inflammatory activity (T-Cell Proliferation).

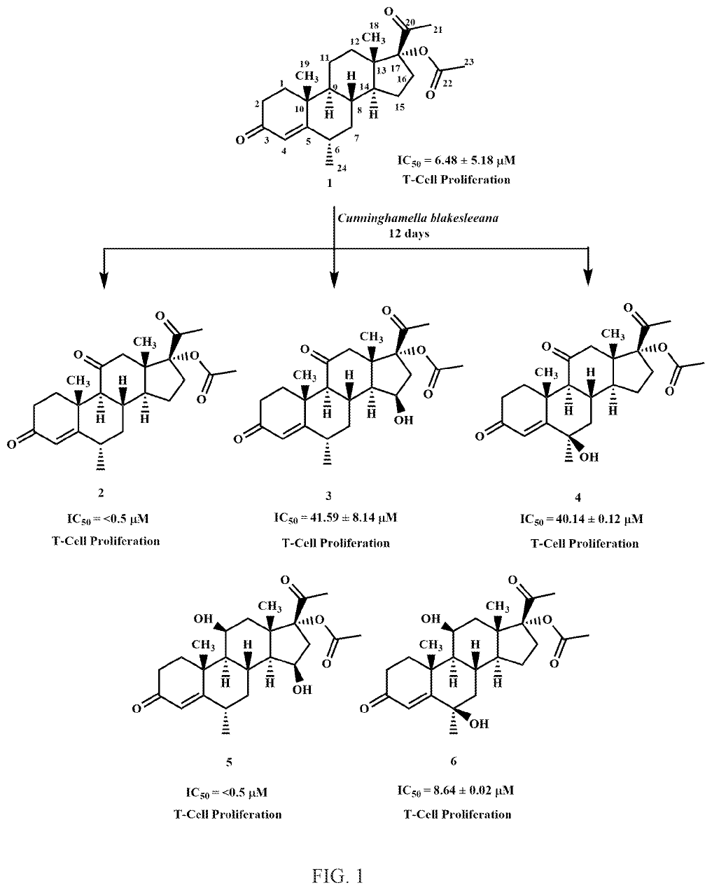

FIG. 2 showed the established binding modes of MPA (a), metabolite 6 (b) in the interface site of human tumor necrosis factor α (TNF-α). The grey carbon sticks show the active site, while the coloured carbon sticks depict ligand. For other atoms, standard color palette was recruited.

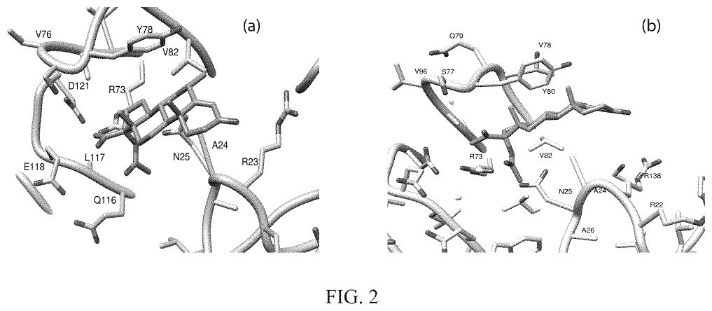

### DETAILED DESCRIPTION OF THE INVENTION

### Microorganisms and Culture Conditions

Fungal cultures of C. blakesleeana (ATCC 8688a) was grown on Sabouraud dextrose agar at 25° C. and stored at 4° C. Glucose (60.0 g), glycerol (60.0 mL), peptone (30.0 g), yeast extract (30.0 g), KH₂PO₄ (30.0 g), and NaCl (30.0 g) were mixed into distilled H₂O (6.0 L) to prepare the media for C. blakesleeana.

### Fermentation of Medroxyprogesterone acetate (1) with Cunninghamella blakesleeana (ATCC 8688)

Compound 1 (0.9 g/60 mL acetone) was distributed among 60 flasks containing 4-day-old culture of C. blakesleeana and kept for fermentation for 10 days. A brown gummy material (1.0 g), obtained after filtration, extraction, and evaporation, was subjected to column chromatography over silica gel for fractionation with increasing polarity of ethyl acetate in petroleum ether. Four main fractions (MPA-1-5) were obtained which were purified on HPLC. Fraction MPA-1 was subjected to recycling RP-HPLC (L-80, MeOH: H₂O=4:1, 4 mL/min) to afford pure compound 2 (8 mg, Rt: 26 min). Compounds 3 (5 mg, Rt: 26 min), and 4 (9 mg, Rt: 22 min) were obtained from fraction MPA-2 and MPA-3 by using recycling RP-HPLC (L80, ACN: H₂O=2:1, 4 mL/min). Similarly, fraction MPA-4 yielded compounds 5 (10 mg, Rt: 28 min), and 6 (26 mg, Rt: 26 min) by using recycling RP-HPLC (L80, MeOH: H₂O=2:1, 4 mL/min).

### 17α-Acetoxy-6α-methylpregn-4-ene-3,11,20-trione (2)

White solid: m. p.: 238-240° C., [α]_{D}²⁵=−91 (c=0.1, MeOH); UV (MeOH) λₘₐₓ nm (log ∈): 237 (6.3); IR (KBr) vₘₐₓ cm⁻¹: 1733, 1707 (C═O), 1674 (C═C—C═O); ¹H-NMR (CH₃OD, 300 MHz), H₂-1 (2.64, m: 1.87, m), H₂-2 (2.74, m; 2.24, m), H-4 (5.75 s), H-6 (2.53, m), H₂-7 (2.00, m; 1.09, m), H-8 (2.09, m), H-9 (2.23, m), H₂-12 (3.06, d, J_{12β,,12α}=12.3; 2.17, d, J_{12α,,12β}=12.3), H-14 (2.39, m), H₂-15 (1.90, m; 1.47, m), H₂-16 (2.94, m; 1.93, m), H₃-18 (0.60, s), H₃-19 (1.40, s), H₃-21 (2.01, s), H₃-23 (2.10, s), H₃-24 (1.10, d, J_{21,6β}=6.6); ¹³C-NMR (CH₃OD, 75 MHz): C-1 (35.7), C-2 (34.2), C-3 (202.6), C-4 (122.2), C-5 (175.7), C-6 (34.6), C-7 (42.2), C-8 (37.3), C-9 (63.3), C-10 (39.8), C-11 (210.9), C-12 (51.6), C-13 (50.8), C-14 (51.4), C-15 (51,5), C-16 (31.7), C-17 (96.5), C-18 (18.6), C-19 (15.7), C-20 (205.5), C-21 (26.8), C-22 (172.4), C-23 (20.9), C-24 (18.2); HREI-MS m/z (mol. formula, calcd value): 400.2247 (C₂₄H₃₂O₅, 400.2250).

### 17α-Acetoxy-15β-hydroxy-6α-methylpregn-4-ene-3,11,20-trione (3)

White solid: m. p.: 265-268° C., [α]_{D}²⁵=−45 (c=0.1, CHCl₃); UV (CHCl₃) λₘₐₓ nm (log ∈): 247 (5.9); IR (CHCl₃) vₘₐₓ cm⁻¹: 3443 (OH), 1736, 1710 (C═O); ¹H-NMR (CH₃OD, 300 MHz), H₂-1 (2.64, m: 1.75, m), H₂-2 (2.44, m; 2.24, m), H-4 (6.00, s), H-6 (2.53, m), H₂-7 (2.28, m; 2.12, m), H-8 (2.42, m), H-9 (2.27, m), H₂-12 (3.07, d, J_{12β,,12α}=12.0; 2.11, d, J_{12α,,12β}=12.0), H-14 (2.30, m), H₂-15 (4.38, m; 1.47, m), H₂-16 (2.95, m; 2.45, m), H₃-18 (0.84, s), H₃-19 (1.45 s), H₃-21 (2.02, s), H₃-23 (2.09, s), H₃-24 (1.11, d, J_{21,6β}=6.6); ¹³C-NMR (CH₃OD, 75 MHz): C-1 (35.8), C-2 (34.2), C-3 (202.6), C-4 (122.2), C-5 (175.3), C-6 (34.6), C-7 (41.2), C-8 (33.4), C-9 (63.4), C-10 (39.9), C-11(210.6), C-12 (52.3), C-13 (50.4), C-14 (55.4), C-15 (68.9), C-16 (44.1), C-17 (96.3), C-18 (18.2), C-19 (18.5), C-20 (204.9), C-21 (26.7), C-22 (172.4), C-23 (20.9), C-24 (18.5). HRESI-MS m/z 417.2304 [M+H]⁺ (C₂₄H₃₃O₆+H requires 417.2277).

### 17α-Acetoxy-6β-hydroxy-6α-methylpregn-4-ene-3,11,20-trione (4)

Colorless solid: m. p: 232-233° C., [α]_{D}²⁵=−175 (c=0.2, CHCl₃); UV (MeOH) λₘₐₓ nm (log ∈): 231 (5.8); IR (MeOH) vₘₐₓ cm⁻¹: 3505 (OH), 1728 (C═O), 1679 (C═C—C═O); ¹H-NMR (CH₃OD, 300 MHz), H₂-1 (2.78, m: 1.68, m), H₂-2 (2.60, m; 2.27, m), H-4 (6.00 s), H₂-7 (1.98, m; 1.45, m), H-8 (2.38, m), H-9 (2.16, m), H₂-12 (3.07, d, J_{12β,,12α}=12.6; 2.19, d, J_{12α,,12β}=12.0), H-14 (2.39, m), H₂-15 (1.93, m; 1.48, m), H₂-16 (2.95, m; 1.94, m), H₃-18 (0.63, s), H₃-19 (1.60, s), H₃-21 (2.11, s), H₃-23 (2.03, s), H₃-24 (1.39, s); ¹³C-NMR (CH₃OD, 75 MHz): C-1 (37.3), C-2 (34.4), C-3 (202.7), C-4 (124.0), C-5 (172.3), C-6 (71.7.), C-7 (46.9), C-8 (32.7), C-9 (62.9), C-10 (39.6), C-11(210.8), C-12 (51.7), C-13 (50.7), C-14 (50.9), C-15 (24.3), C-16 (31.7), C-17 (96.6), C-18 (19.9), C-19 (15.7), C-20 (205.5), C-21 (26.8), C-22 (172.4), C-23 (20.9), C-24 (18.6); HREI-MS m/z (mol. formula, calcd value): 416.2201 (C₂₄H₃₂O₆, 416.2199).

### 17α-Acetoxy-11β,15β-dihydroxy-6α-methylpregn-4-ene-3,20-dione (5)

Colorless solid: m. p. 228-230° C., [α]_{D}²⁵=+100 (c=0.1, MeOH); UV (CHCl₃) λₘₐₓ nm (log ∈): 248 (5.8); IR (CHCl₃) vₘₐₓ cm⁻¹: 3463 (OH), 1732 (C═O), 1656 (C═C—C═O); ¹H-NMR (CH₃OD, 300 MHz): H₂-1 (2.17, m; 1.94, m), H₂-2 (2.47, m; 2.34, m), H-4 (5.69, br d, J_{4,,12β}=1.5 Hz), H-6 (2.66, m), H₂-7 (2.33, m; 0.88, m), H-8 (2.50, m), H-9 (1.13, m), H-11 (4.40, br. d, J_{11α,,12β}=3.0), H₂-12 (2.13, m; 1.71, dd, J_{12β,,12α}=2.7, J_{12β,,11α}=13.5), H-14 (1.61, dd, J_{14α,,18β}=5.7, J_{14α,,15β}=11.4), H-15 (4.30, m), H₂-16 (2.89, dd, J_{16β,16α}=2.1, J_{16β,15α}=16.6; 2.27, m), H₃-18 (1.13, s), H₃-19 (1.48, s), H₃-21 (2.02, s), H₃-23 (2.04, s), H₃-24 (1.08, d, J_{21,6β}=6.0); ¹³C-NMR (CH₃OD, 75 MHz): C-1 (35.9), C-2 (34.4), C-3 (202.7), C-4 (119.6), C-5 (179.6), C-6 (34.5), C-7 (42.5), C-8 (28.9), C-9 (57.4), C-10 (41.1), C-11 (68.5), C-12 (41.1), C-13 (47.2), C-14 (57.9), C-15 (69.9), C-16 (43.3), C-17 (97.8), C-18 (20.2), C-19 (22.5), C-20 (205.5), C-21 (26.7), C-22 (172.6), C-23 (20.9), C-24 (18.6); HRESI-MS m/z: 419.2407 [M+H]⁺ (C₂₄H₃₅O₆+H requires 419.2433).

### 17α-Acetoxy-11β,6β-dihydroxy-6α-methylpregn-4-ene-3,20-dione (6)

Colorless solid: m. p.: 152-154° C., [α]_{D}²⁵=−65 (c=0.1, CHCl₃); UV (MeOH) λₘₐₓ nm (log ∈): 247 (5.9); IR (CHCl₃) vₘₐₓ cm⁻¹: 3439 (OH), 1730 (C═O), 1661 (C═C—C═O); ¹H-NMR (CH₃OD, 300 MHz), H₂-1 (2.13, m; 1.83, m), H₂-2 (1.80, m; 1.43, m), H-4 (5.95, s), H₂-7 (2.04, m; 1.21, m), H-8 (2.37, m), H-9 (1.01, dd, J_{9α,11β}=3.4, J_{9α,8β}=11.4), H-11 (4.40, br. d, J_{11α,,12β}=2.7), H₂-12 (2.13, dd, J_{12α,,12β}=3.6, J_{12α,,11α}=13.8, 1.76, m), H-14 (1.72, m), H₂-15 (1.80, m; 1.43, m), H₂-16 (2.90, m; 1.71, m), H₃-18 (0.90, s), H₃-19 (1.63, s), H₃-21 (2.03, s), H₃-23 (2.06, s), H₃-24 (2.06, s); ¹³C-NMR (CH₃OD, 75 MHz): C-1 (38.5), C-2 (34.5), C-3 (203.5), C-4 (122.5), C-5 (174.7), C-6 (71.5), C-7 (47.7), C-8 (29.0), C-9 (57.4), C-10 (40.6), C-11 (68.2), C-12 (41.2), C-13 (47.4), C-14 (53.9), C-15 (24.8), C-16 (31.1), C-17 (97.9), C-18 (17.2), C-19 (23.5), C-20 (206.2), C-21 (26.8), C-22 (172.6), C-23 (21.0), C-24 (29.3); HRESI-MS m/z: 419.2415 [M+H]⁺ (C₂₄H₃₅O₆, 419.2433).

T-Cell Proliferation Assay

The T-cell proliferation inhibitory activity of compounds 2-6 were evaluated by following the method of Nielsen et al. (1998). Peripheral blood mononuclear cells (PBMC) were isolated by Ficoll-Hypaque gradient centrifugation from the blood of healthy human volunteers. The concentration of the cell was adjusted to 2×10⁶ cells/mL in RPMI-1640 media containing 5% FBS (Fetal bovine serum) and then 5 μg/mL phytohemagglutinin (PHA) was added into each well of a sterile 96-well plates. Different concentrations of test compounds (0.2, 1, 5, and 25 μg/mL) were then added, each in triplicate. Positive control wells contained cells and PHA, whereas the negative control contains cells alone. The plate was incubated in 5% CO₂ at 37° C. for 72 hours and the cells were pulsed with 25 μL of tritiatedthymidine (0.5 μci/well), and the incubation was continued for 18 hours. Cells were harvested on a glass fiber filter, and the effect of the test compounds on proliferation was evaluated using a LS65000 liquid scintillation counter (Beckman Coulter, Fullerton, Calif., USA) quantitatively using a LS65000 liquid scintillation counter (Beckman Coulter, CA, USA). Counts per minute (CPM) were recorded, and the inhibiton of T-cell proliferation was calculated in comparison to control containing cells and PHA.

### Molecular Docking

To establish the binding mode of medroxyprogesterone acetate (MPA) and the bio-transformed derivatives, molecular docking simulations were carried out using Vina. In case of human TNF-a, the crystal coordinates of the protein were retrieved under the accession number 2AZ5 from RCSB Protein Data Bank [Burley et al., Nucleic Acids Res. 2021, 8, 49]. TNF-α is homotrimer, with active site lying in the interface of chains A and B [Zia K et. al., Sci. Rep. 2020, 10, 20974], thus chain C was removed. Following the conformational flexibility, the structure lacks coordinates of several loops, which were added using the loop modeler algorithm implemented in MODELLER. Following the verification of the coordinates, the structure was converted into PDBQT format using Auto Dock Tools (ADT) interface. Using ADT, missing hydrogens were added, and gasteiger charges were assigned.

The ligands were sketched using Chemdraw, and saved in MOL format (2D), which were then converted to three dimensional coordinates using Obabel [O'Boyle et al., J. Cheminformatics, 2011, 3, 33]. Using ADT, the ligands were assigned Kollman charges, and their roots were autodetected for conversion in PDB QT format.

For the definition of grid space, a grid box of 40×40×40 Å was developed based on the coordinates of the cognate ligand, using autogrid4. The coordinates of the search space (−9.57, 67.486, 20.528) were then employed in autodock Vina [Trott O et al., J Comput Chem. 2010, 31, 455] to define the search space. In Vina, the seeds were generated randomly, and global search exhaustiveness was employed to find the best possible. A total of nine poses were generated for each ligand, with a maximum allowed difference of 3 kcal/mol. The top-ranked pose of each ligand was retrieved and analyzed visually in UCSF Chimera [Pettersen et al., J Comput Chem. 2004. 25. 1605].

### Results and Discussion

Medroxyprogesterone acetate (1) was isolated from a drug Medrosterona (Seignior Pharma, Pakistan). Purity was checked on TLC and the structure was established on the basis of spectroscopic techniques. Biotransformation of medroxyprogesterone acetate (MPA) (1) was carried out with Cunninghamella blakesleeana (ATCC 8688). This yielded five new metabolites. These metabolites were purified by high performance liquid chromatography (HPLC), and characterized as 17α-acetoxy-6α-methylpregn-4-ene-3,11,20-trione (2), 17α-acetoxy-15β-hydroxy-6α-methylpregn-4-ene-3,11,20-trione (3), 17α-acetoxy-60-hydroxy-6α-methylpregn-4-ene-3,11,20-trione (4), 17α-acetoxy-11β,15β-dihydroxy-6α-methylpregn-4-ene-3,20-dione (5), and 17α-acetoxy-6β,11β-dihydroxy-6α-methylpregn-4-ene-3,20-dione (6) by using modern spectroscopic techniques. In T-cell proliferation assay compounds 2, and 5 with IC₅₀<0.5 and 6 with IC₅₀=8.64±0.02 μM were found potent inhibitors as compared to substrate (1) (IC₅₀=6.48±5.18) and standard prednisolone (IC₅₀=9.75±0.03 μM).

The HREI-MS of metabolite 2 showed M⁺ at m/z 400.2247 (C₂₄H₃₂O₅). ¹H-NMR spectrum showed a downfield shift of H-9 at δ 2.23, and two doublets of H-12 at δ 3.06 and 2.17, and indicated the oxidation at C-11. The ¹³C-NMR spectrum of metabolite 2 showed an additional new quaternary carbon at δ 210.9. The ²J HMBC correlations of H-9 and H-12 with C-11 supporting the location of newly formed ketone functionality at C-11. The structure was characterized as 17α-acetoxy-6α-methylpregn-4-ene-3,11,20-trione (2) as a new metabolite

The ESI-MS (+) of metabolite 3 exhibited the M⁺ at m/z 417.2304 [M+H]⁺ (C₂₄H₃₃O₆+H requires 417.2277), 30 a.m.u. higher than the substrate 1 and indicating the oxidation of substrate. The ¹H-NMR spectrum was different from the substrate in two aspects: first a downfield shift of H-9, and two doublets of H-12. These informations indicated oxidation at C-11. Secondly, the downfield methine signal at δ 4.38 for H-15. The ¹³C-NMR spectrum showed an additional new quaternary carbon at δ 210.6. In the HMBC spectrum, ²J correlations of H-9 and H-12 with C-11 further supported a ketone functionality at C-11. Furthermore, ³J correlations of H-15 with C-17 and C-13 supported an OH at C-15. The stereochemistry of newly introduced OH at C-15 was deduced to be β (axial) based on NOESY correlation between H-15 and H-14. The structure was thus characterized as 17α-acetoxy-15β-hydroxy-6α-methylpregn-4-ene-3,11,20-trione (3) as a new metabolite.

The EI-MS of metabolite 4 exhibited the M⁺ at m/z at 416.2201. The ¹H-NMR spectrum of metabolite 4 was different from the substrate in two aspects: first the downfield shift of H-9, and two doublets of H-12 Showing correlation with C-11. Secondly, the absence of a doublet for CH₃-24 indicated the hydroxylation at C-6. The ¹³C-NMR spectrum showed a new quaternary carbon at C-11. The new quaternary carbon located on the basis of downfield shift of C-9, and C-12. ²J HMBC correlations of H-9 and H-12 with C-11 further supported a ketonic functionality at C-11. Whereas, the ³J correlation of H-19 with C-9 supported a OH at C-11. ²J and ³J HMBC correlations of H-7 and H-4 with C-6 respectively, supported the hydroxylation at C-6. The structure was thus characterized as 17α-acetoxy-60-hydroxy-6α-methylpregn-4-ene-3,11,20-trione (4) as a new metabolite.

The ESI-MS of compound 5 exhibited the M⁺ at 419.2407 [M+H]⁺ (C₂₄H₃₅O₆+H requires 419.2433). The ¹H-NMR spectrum of 5 was different from the substrate 1 due to appearance of two new downfield methine signal at δ 4.40, and 4.30. The ¹³C-NMR spectrum showed two additional hydroxyl-bearing methine carbons i.e. C-11 and C-15. The location of C-11 OH was deduced on the basis of downfield shift of C-9 and C-12. Similarly, C-15 OH was deduced based on downfield shifts of C-14, and C-16. The assigned position OH at C-11 was further deduced by the ³J HMBC correlations with C-13 and C-8. The assigned position of new OH at C-15 was further confirmed by the ³J HMBC correlations of H-15 with C-13 and C-17. The stereochemistry of newly introduced OH at C-11 was also deduced to be β (axial) based on NOESY correlation between H-11 and H-9 and similarly, at C-15 was deduced to be β (axial) on the basis of NOESY correlation between H-15 and H-14. The new structure of metabolite 5 was characterized as 17α-acetoxy-11β-hydroxy-15β-hydroxy-6α-methylpregn-4-ene-3,20-dione.

Metabolite 6 showed ESI-MS exhibited the M⁺ at m/z 419.2419 [M+H]⁺ (C₂₄H₃₅O₆+H requires 419.2433), which is 32 a.m.u. higher than the substrate 1, indicating oxidation of substrate 1. The ¹H-NMR spectrum of metabolite 6 was different from the substrate 1 in two aspects: first a new downfield methine signal in the spectrum of compound 6 at δ 4.40. Secondly, the appearance of CH₃-24 as a singlet, instead of doublet indicating an OH at C-6. The ¹³C-NMR spectrum showed additional OH bearing methane was at C-11. The location newly OH at C-11 was based on downfield shift of neighboring C-9 and C-12. ²J HMBC correlation of H-11 with C-9, and ³J correlations with C-8, and C-13. The stereochemistry of newly introduced hydroxyl group at C-11 was deduced to be β (axial) on the basis of NOESY correlation between H-11 and H-9 and H-14 (δ 1.72). The new structure of metabolite 6 was characterized as 17α-acetoxy-11β,6β-dihydroxy-6α-methylpregn-4-ene-3,20-dione.

In adaptive immunity, the T-cells are of central importance. The control the activation and proliferation of other immune cells, including B-cells, macrophages, and dendritic cells through secretion of various cytokines and regulating the humoral and cellular immune responses. They are also involved in the pathogenesis of various chronic inflammatory and autoimmune diseases. Hence for the treatment of ailments due to dysregulated immune responses, the inhibition of T-cells proliferation provides promising approach. During this study, compounds 2 and 5 were found to be potent inhibitors against T-cells than medroxyprogesterone acetate (1) and standard prednisolone. The increased potency of compound 2 may be due C-11 ketone functionality, while in compound 5, this may be due to β OH at C-11 and C-15. Furthermore, compound 6 showed a strong activity while compounds 3 and 4 were found to be moderately active as compared to substrate (1) and standard prednisolone in vitro.

Molecular docking studies were carried out to establish the protein-ligand contact profiles. Human TNF-α is homotrimer, requiring effective dimerization to activate downstream signaling pathways. Thus, the dimerization site is often recruited by the inhibitors. Docking studies suggest that medroxyprogesterone acetate (MPA) develops a complex by binding effectively at the interface site (ΔG-5.03 kcal/mol). The progesterone ring of the compounds exhibits hydrophobic contacts with the surrounding residues; L117, Y78, and V82. Analysis of binding energy as well as contact profile suggest that the substrate because of higher lipophilic character establish a stacking position at the dimerization site, effectively hindering the dimerization by recruiting crucial residues.

What is claimed is:

1. A method of treatment of chronic inflammations due to proliferation of T-cells, comprising on administration of an effective amount of newly developed anti-inflammatory agents having formulae 2-6 or their isomers, salts or solvates, or co-crystals in suitable pharmaceutical excipients, adjuvant, carrier, or diluent to humans, and animals in need thereof.

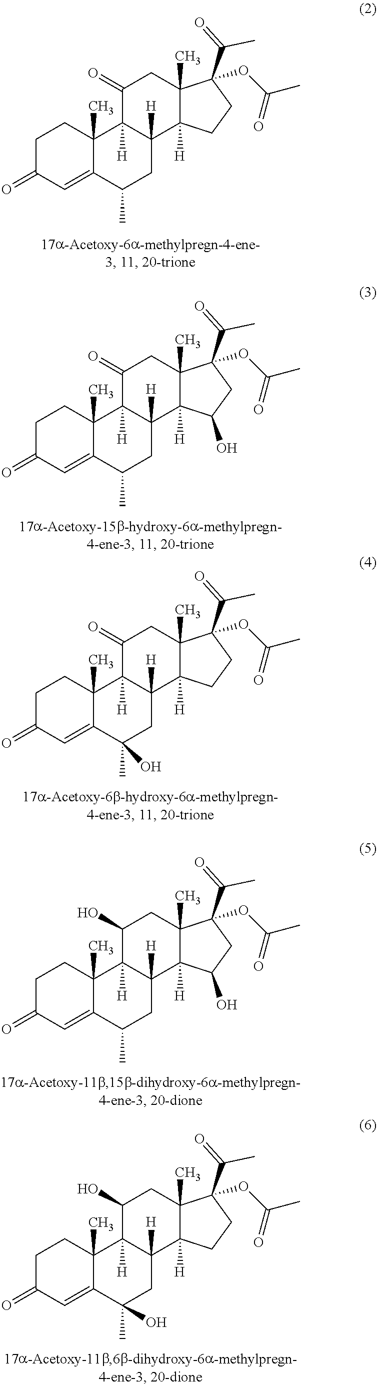

2. Formulae 2-6 as in claim 1, have the potential to inhibit cellular immune responses and might be useful in suppressing various chronic inflammatory and autoimmune disorders.

3. Formulae 2-6 as in claim 1, can be synthesized by biotransformation of medroxyprogesterone acetate (1) or through the chemical synthesis.
